(19) United States
(12) Patent Application Publication
(10) Pub. No.: US 2020/0061585 A1
(43) Pub. Date: Feb. 27, 2020

(54) IMPROVED PROCESS TO DEPOSIT PD- NANOPARTICLES

(71) Applicant: DSM IP ASSETS B.V., Heerlen (NL)

(72) Inventors: Roman GOY, Kaiseraugst (CH); Jonathan Alan MEDLOCK, Kaiseraugst (CH)

(21) Appl. No.: 16/609,591

(22) PCT Filed: May 1, 2018
(86) PCT No.: PCT/EP2018/061066

§ 371 Date: Oct. 30, 2019

(30) Foreign Application Priority Data
May 1, 2017 (EP) 17168889.8

(51) Int. Cl. B01J 23/44; B01J 23/10; B01J 21/06; B01J 23/02; B01J 35/00; B01J 37/34; B01J 37/02; C07C 29/17

(52) U.S. Cl. CPC B01J 23/44; B01J 23/10; B01J 21/063; B01J 21/066; C07C 29/17; B01J 35/0013; B01J 37/343; B01J 37/0221; B01J 37/0219; B01J 23/02

(57) ABSTRACT

The present invention relates to an improved process to prepare and deposit Pd-nanoparticles onto a metal oxide.

The present invention relates to an improved process to prepare and deposit Pd-nanoparticles onto a metal oxide.

Catalyst with Pd nanoparticles are very well-known and widely used catalyst.

A very prominent species of these kind of catalyst is the so called Lindlar catalyst.

The Lindlar catalyst is a heterogeneous catalyst which consists of palladium deposited on a calcium carbonate carrier which is also treated with various forms of lead.

There are other species of similar catalysts, wherein only palladium nanoparticles are deposited and which are lead free.

There are methods known how to deposit (=to dope) a metal oxide (which is part of a catalytic system) with Pd-nanoparticles.

The deposition methods known from the prior art have disadvantages like for example:
The Pd-nanoparticles formed by reduction with H₂ (which is the usual and common way) are not well-defined nanoparticles, regarding size and shape.
For H₂ reduction, H₂-gas is bubbling through the Pd-salt solution, which means a large excess of reducing agent is used,
For the H₂-method, PdCl₂ is used as Pd-source. To dissolve this salt in water, Na₂MoO₄ is needed to form a water-soluble Pd-complex, which means longer preparation time and the loading of Molybdenum onto the catalyst surface. Using the other Pd-salt does not work as well and vice versa.

It was now found that when the process of depositing Pd-nanoparticles comprises a sonication step, these disadvantages are overcome.

Therefore, the present invention relates to a process for depositing Pd-nanoparticles on a metal oxide (or a mixture of metal oxides), wherein the process comprises a sonication step.

Furthermore, it was found that when the Pd-salt solution comprises a surfactant, these disadvantages are overcome, too.

Therefore, the present invention relates to a process for depositing Pd-nanoparticles on a metal oxide (or a mixture of metal oxides), wherein the process comprises a sonication step as well as a surfactant.

The advantages of the new process are for example that
the Pd-nanoparticles which are formed by using the new process are almost spherical and well defined regarding size
no H₂ gas is used.
it is a very fast and efficient process.

The doped palladium nanoparticles can be isolated from each other on the surface, or can also be agglomerated forming clusters of palladium nanoparticles of varying sizes.

The metal oxide, which is doped by the Pd-nanoparticles can be in powder form (or other solid form) or it can be that the metal oxide is used as a layer, which is used to coat another material. It can be a metal oxide (from one metal) as well as mixture of various metal oxides.

Sonication is an essential part of the process according to this invention.

Sonication is the act of applying sound energy to agitate particles in a sample. Ultrasonic frequencies (>20 kHz) are usually used, leading to the process also being known as ultrasonication or ultra-sonication.

It is usually applied using an ultrasonic bath or an ultrasonic probe.

The process according to the present invention comprises usually (and preferably) the following steps:
(a) preparing an aqueous solution of Pd-salt optionally adding a polyethylene glycol
(b) heating the solution of step (a) and subjecting the solution to sonication
(c) adding a reducing agent, preferably a solution of formate, to the Pd solution
(d) adding the metal oxide powder
(e) the suspension which is obtained in step (d) is filtrated and dried

It results a powder which has excellent properties as a catalyst.

### Step (a)

The Pd salt is dissolved in water (or aqueous solvent, which means that water is mixed at least one other solvent).

Any commonly known and used Pd-salt can be used. Suitable salts are PdCl₂ or Na₂PdCl₄. It can be one Pd-salt as well as a mixture of two or more Pd-salts. Furthermore, it is of an advantage to add at least one surfactant to the solution. Suitable are i.e. polyethylene glycol (PEG), polyvinylpyrrolidones (PVP) or glucosamides.

### Step (b)

The solution of step is usually heated up to elevated temperature. Usually not to a higher temperature as the boiling point of the solvent (or solvent mixture used). Usually it is heated up to a temperature of between 30-80° C.

The sonication is usually carried out at a frequency of 30-50 kHz.

The duration of the sonication step is usually at least 10 minutes, preferred more than 20 (suitable and preferred range is 30-120 minutes). The maximal length of the duration of the sonication step is not critical.

The sonication step can be carried out by using an ultrasonic bath or an immersion probe. Or even a combination of both methods is possible.

### Step (c)

To the solution of step (b) a reducing agent is added. Usually it is a sodium formate solution. But also, other formate salts (or mixtures of formate salts) could be used. Optionally (instead of or additionally), it is also possible to add H₂-gas, L-ascorbic acid, and/or formic acid.

### Step (d)

To the solution of step (c) the metal oxide powder (or a mixture of metal oxide powders) are added. Usually the reaction mixture is stirred.

### Step (e)

Finally, the suspension of step (d) is filtered and the obtained doped metal oxide powder is usually washed and dried.

It is clear, that some of the steps can be carried out several times. It is for example possible that the sonication also takes place in other steps than only in step (b) The so obtained catalysts are then activated before use.

The following examples serve to illustrate the invention. All percentages are related to weight and the temperatures are given in degree Celsius, if not otherwise stated.

### EXAMPLES

### Preparation of Oxide Powder Catalyst

Sodium tetrachloropalladate(II) (0.48 mmol) was dissolved in 133 mL of Millipore water and PEG-MS40 (3.2 mmol) was added. The solution was heated to 60° C. and sonication was started at this temperature. A freshly prepared solution of sodium formate (16 mM, 67 mL) were added. The solution was sonicated for further 60 minutes at this temperature and then cooled to room temperature followed by addition of the desired oxide powder.

The following commercially available mixed oxides from Sasol Performance Chemical have been used:

PURALOX®SCFa-160/Ce20 (81.0% Al₂O₃/19.0% CeO₂)
PURALOX®TH 100/150 Ti 10 (89.6% Al₂O₃/10.4% TiO₂)
PURALOX®SCFa-190 Zr20 (78.8% Al₂O₃/21.2% ZrO₂)
PURALOX®Mg28/100 (71.2% Al₂O₃/28.8% MgO)
Results from Selective Semi-Hydrogenation of an Alkyne to an Alkene

In a typical hydrogenation experiment 40.0 g of 2-methyl-3-butyne-2-ol (MBY), the desired amount of oxide powder catalyst as well as 6 mg sulfur-containing catalyst poison/mg_{Pd} were added to a 125 mL autoclave reactor. Isothermal conditions during the hydrogenation reaction (338 K) were maintained by a heating/cooling jacket. The reactor was equipped with a gas-entrainment stirrer. Pure hydrogen was supplied at the required value under nitrogen atmosphere. After purging with nitrogen, the reactor was purged with hydrogen and heated to the desired temperature. The pressure in the reactor (3.0 bar) was maintained during the experiments by supplying hydrogen from external reservoir. The reaction mixture was stirred with 1000 rpm. Liquid samples (200 μL) were periodically withdrawn from the reactor starting at a minimum conversion of 95% of MBY and analysed by gas-chromatography (HP 6890 series, GC-system). Selectivity is reported as amount of the desired semi-hydrogenation product (2-methyl-3-butene-2-ol (MBE)) compared to all reaction products.

TABLE 1
results of the hydrogenation (example 1 is a comparison test with a
commercially available Lindlar catalyst)
Reaction conditions: 40.0 MBY, 1000 rpm, 3.0 bar H₂, 65° C.,
6 mg sulfur-containing catalyst poison/mg_{Pd}

| | | Pd-loading | | | | |
|---|---|---|---|---|---|---|
| | Oxide | (wt %) and | Conv. | Time | Select. | Activity |
| Exp. | powder | cat. amount | (%) | (min) | (%) | (molh⁻¹g⁻¹_{Pd}) |
| 1^{a} | CaCO₃ | 5.00 Pd | >99.9 | 156 | 95.6 | 275.6 |
| | | (12 mg) | | | | |
| 2 | CeO₂/ | 0.50 | >99.9 | 108 | 96.9 | 193.8 |
| | ZnO | (250 mg) | | | | |
| 3 | Al₂O₃/ | 1.00 | >99.9 | 168 | 95.3 | 166.6 |
| | MgO | (100 mg) | | | | |
| 4 | Al₂O₃/ | 1.00 | 99.9 | 186 | 95.9 | 136.1 |
| | TiO₂ | (100 mg) | | | | |
| 5 | Al₂O₃/ | 1.00 | 99.9 | 195 | 95.8 | 133.3 |
| | CeO₂ | (100 mg) | | | | |
| 6 | Al₂O₃/ | 1.00 | 99.9 | 220 | 95.8 | 118.0 |
| | ZrO₂ | (100 mg) | | | | |

^{a}5% Pd on CaCO₃ was obtained from Evonik.

It can be seen, that the catalysts produced by using the new method show better selectivity.

1. A process for depositing Pd-nanoparticles on at least one metal oxide, wherein the process comprises a sonication step.

2. Process according to claim 1, wherein the at least one metal oxide in powder form (or other solid form) or in the form of a layer, which is used to coat another material.

3. Process according to claim 1, wherein PdCl₂ and/or Na₂PdCl₄.

4. Process according to claim 1, wherein the sonication is carried out at a frequency of 30-50 kHz.

5. Process according to claim 1, wherein the sonication is carried out by using an ultrasonic bath and/or an immersion probe.

6. Process according to claim 1, wherein at least one reducing agent (preferably sodium formate) is added to the Pd-salt solution.

7. Process according to claim 1, wherein at least one surfactant (preferably a polyethylene glycol) is added to the Pd-salt solution.
